(19) United States
(12) Patent Application Publication
(10) Pub. No.: US 2022/0319698 A1
(43) Pub. Date: Oct. 6, 2022

(54) SYSTEM AND METHOD FOR GENERATING A RATION PROTOCOL AND INSTITUTING A DESIRED ENDOCRINAL CHANGE

(71) Applicant: KPN INNOVATIONS, LLC., Lakewood, CO (US)

(72) Inventor: Kenneth Neumann, LAKEWOOD, CO (US)

(73) Assignee: KPN INNOVATIONS, LLC., Lakewood, CO (US)

(21) Appl. No.: 17/221,418

(22) Filed: Apr. 2, 2021

(51) Int. Cl. G16H 50/20; G16H 20/60; G06N 3/04

(52) U.S. Cl. CPC G16H 50/20; G16H 20/60; G06N 3/04

(57) ABSTRACT

In an aspect, system and methods for generating a ration protocol for instituting a desired endocrinal change include receiving at least an endocrinal representation and a ration record, generating at least a change of nutrition by receiving training data correlating nutritional elements to endocrinal representations, training a machine learning model as a function of a machine learning algorithm and the training data, and generating at least a change of nutrition as a function of the machine learning model, and the ration record, and generating the ration protocol as a function of the at least a change of nutrition.

### FIELD OF THE INVENTION

The present invention generally relates to the field of AI and simulation/modeling. In particular, the present invention is directed to a system and method for generating a ration protocol and instituting a desired endocrinal change.

### BACKGROUND

Endocrinal changes in humans are known to be affected by nutrition. However, dietary changes can result in endocrinal changes, which are multifactorial, complex, unique to an individual, and unpredictable. As a result, only extremely simple diets can thus far be predicted to result in reliable endocrinal changes to a subject.

### SUMMARY OF THE DISCLOSURE

In an aspect, a method of generating a ration protocol for instituting a desired endocrinal change includes receiving, using a computing device, at least an endocrinal representation and a ration record, generating, using the computing device and the ration record, at least a change of nutrition, where generating the at least a change of nutrition, additionally includes receiving training data correlating nutritional elements to endocrinal representations, training a machine learning model as a function of a machine learning algorithm and the training data, and generating at least a change of nutrition as a function of the machine learning model, and the ration record, and outputting, using the computing device, the ration protocol as a function of the at least a change of nutrition.

In another aspect, a system for generating a ration protocol for instituting a desired endocrinal change includes a computing device configured to receive at least an endocrinal representation and a ration record, generate, using the ration record, at least a change of nutrition, where generating the at least a change of nutrition, additionally includes receiving training data correlating nutritional elements to endocrinal representations, training a machine learning model as a function of a machine learning algorithm and the training data, and generating at least a change of nutrition as a function of the machine learning model, and the ration record, and output the ration protocol as a function of the at least a change of nutrition.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of a system for generating a ration protocol and instituting a desired endocrinal change;

FIG. 2 is a block diagram of an exemplary embodiment of a machine-learning module;

FIG. 3 is a schematic diagram of an exemplary embodiment of a neural network;

FIG. 4 is a schematic diagram of an exemplary embodiment of a node of a neural network;

FIG. 5 is a flow diagram of an exemplary embodiment of a method for generating a ration protocol and instituting a desired endocrinal change; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating and recommending a ration protocol. In an embodiment, ration protocol may be instituted to induce a desired endocrinal change. In some embodiments, a ration protocol may include multiple rations, or foods, that together are likely to institute a desired endocrinal change. In some cases, a desired endocrinal change may be a forestalling or slowing of an impending or immediate hormonal change, for instance without limitation hormonal changes resulting from menopause.

Aspects of the present disclosure can be used to reliably predict a ration protocol that may be useful in instituting a desired endocrinal change. Aspects of the present disclosure can also be used to determine a desired endocrinal change, by comparing a current endocrinal representation to a normal range.

Aspects of the present disclosure allow for a nutritional changes to be used to predictably affect endocrinal changes. Exemplary embodiments illustrating aspects of the present disclosure are described below in the context of several specific examples.

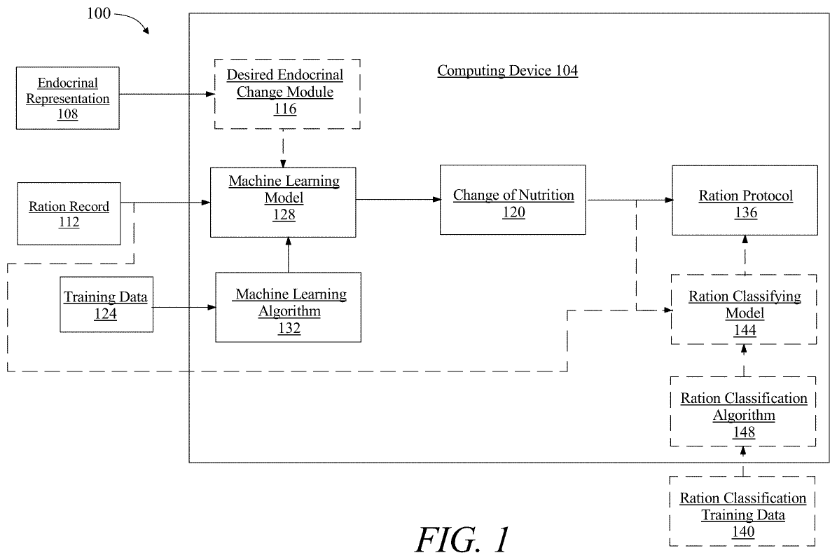

Referring now to FIG. 1, an exemplary embodiment of a system 100 generating a ration protocol for instituting a desired endocrinal change is illustrated. As used in this disclosure a “desired endocrinal change” is a change in at least a hormone within a person, for instance without limitation a change in hormonal level, sensitivity, and/or resistance to one or more of insulin, cortisol, estriol, estradiol, estrone, estrogen, leptin, progesterone, or testosterone. In some cases, a desired endocrinal change refers to change to one or more bioidentical hormones (e.g., estriol, estradiol, estrone, progesterone, testosterone, and the like). In some cases, a desired endocrinal change is desired to counteract a hormonal imbalance. Some non-limiting examples of desired endocrinal changes that may impact a hormonal imbalance include: an increase in free testosterone, a decrease in insulin sensitivity, a decrease in insulin levels, a decrease/increase in cortisol secretion, a change in thyroid hormone regulation, a decrease in leptin sensitivity, and the like. An endocrinal change may be indicated by a change in at least a biomarker, for instance without limitation a measured level of at least a hormone within a bodily tissue or fluid.

Still referring to FIG. 1, system includes a computing device 104. computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, according to some embodiments, computing device is configured to receive at least an endocrinal representation 108. As used in this disclosure an “endocrinal representation” is a datum indicative of hormones within a person, for instance without limitation a hormone level. In some cases, an endocrinal representation may include a biological test result. Non-limiting examples of endocrinal representations include measures of any of the following adiponectin, adrenocorticotropic hormone, agouti-related protein, brain-derived neurotrophic factor, C-peptide, calcitonin, ciliary neurotrophic factor, cortisol, erythropoietin, follicle-stimulating hormone, gastric inhibitory polypeptide, glucagon, glucagon-like peptide 1, granulocyte colony-stimulating factor, growth hormone, human chorionic gonadotropin beta, insulin, insulin-like growth factor binding protein 4, insulin-like growth factor binding protein 5, insulin-like growth factor binding protein 6, insulin-like growth factor I, insulin-like growth factor-binding protein 1, insulin-like growth factor-binding protein 2, insulin-like growth factor-binding protein 3, leptin, leptin receptor, luteinizing hormone, progesterone, proinsulin, intact, total proinsulin, prolactin, resistin, secretin, sex hormone-binding globulin, total testosterone, thrombopoietin, thyroglobulin, thyroglobulin antibody, thyroid-stimulating hormone, thyroxine-binding globulin, and transthyretin.

Still referring to FIG. 1, in some embodiments, computing device 104 is configured to calculate a desired endocrinal change as a function of endocrinal representation. For example in some embodiments, a desired endocrinal change module 116 may be used to calculate desired endocrinal change as a function of endocrinal representation 108, which may be implemented in any manner suitable for implementation of any computing device, module, and/or component of computing device 104 as described above. Modules and/or components described as included in endocrinal representation module 108 are presented for exemplary purposes only; functions and/or structure pertaining to each such module and/or component may be implemented in any alternative or additional manner in computing device 104 and/or any component, module, and/or device incorporated in or communicatively connected to computing device 104, in any manner that may occur to persons skilled in the art, upon reviewing the entirety of this disclosure. In some cases, calculating a desired endocrinal change as a function of an endocrinal representation may include comparing an endocrinal representation with an endocrinal standard. Without limitation, an endocrinal standard may include a normal range of an endocrinal representation. In some cases, a normal range may include a biological reference range having an upper limit and a lower limit; the biological reference range may be based upon measurements from a group of otherwise healthy people. In some cases, normal range may be dependent upon one or more factors, including without limitation age and sex. For example, in some cases an endocrinal representation may include a measurable metric and an endocrinal standard may include a normal range, within which the measurable metric is substantially considered unremarkable. Therefore, in some exemplary embodiments, calculating a desired endocrinal change may additionally include calculating a distance between an endocrinal representation 108 and an endocrinal standard. A “distance,” as used in this disclosure, is a quantitative value indicating a degree of similarity of a set of data values to another set of data values. In some cases, a distance between any two or more metrics, for example an endocrinal representation 108 and an endocrinal standard or a nutritional standard and at least a nutritional element, may be calculated using any method described in detail below.

Still referring to FIG. 1, for instance, and without limitation, an endocrinal representation and an endocrinal standard, may be represented a vector. Each vector may be represented, without limitation, as an n-tuple of values, where n is at least two values. Each value of n-tuple of values may represent a measurement or other quantitative value associated with a given category of data, or attribute, such as an endocrinal measure, examples of which are provided in further detail throughout this disclosure; a vector may be represented, without limitation, in n-dimensional space using an axis per category of value represented in n-tuple of values, such that a vector has a geometric direction characterizing the relative quantities of attributes in the n-tuple as compared to each other. A non-limiting distance may include a degree of vector similarity. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent, for instance as measured using cosine similarity; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values. As a non-limiting illustration, an endocrinal standard, and/or one or more subsets thereof, may be represented using a vector or other data structure, and a plurality of endocrinal representation output from one or more machine-learning processes may be represented by a like data structure, such as another vector; a distance comparing the two data structures may then be calculated and compared to distances calculations to find a minimal distance calculation and/or a set of minimal distance calculations. A set of minimal distance calculations may be a set of distance calculations less than a preconfigured threshold distance from data structure representing a desired endocrinal function. In some cases, one or more machine-learning processes are utilized to prepare plurality of endocrinal representations, using a plurality of candidate inputs, for example without limitation rations, ration protocols 136, and/or at least a nutritional element. Preconfigured threshold may be set by one or more expert users and/or determined statistically, for instance by finding a top quartile and/or number of percentiles of proximity in a series of distance determinations over time for user, at one time for a plurality of users, and/or over time for a plurality of users. Plurality of users may include a plurality of users selected by a user classifier, which may classify user to a plurality of users having similar physiological data and/or user data; implementation of a user classifier may be performed, without limitation, as described in U.S. Nonprovisional application Ser. No. 16/865,740, filed on May 4, 2020 and entitled “METHODS AND SYSTEMS FOR SYSTEM FOR NUTRITIONAL RECOMMENDATION USING ARTIFICIAL INTELLIGENCE ANALYSIS OF IMMUNE IMPACTS,” the entirety of which is incorporated herein by reference.

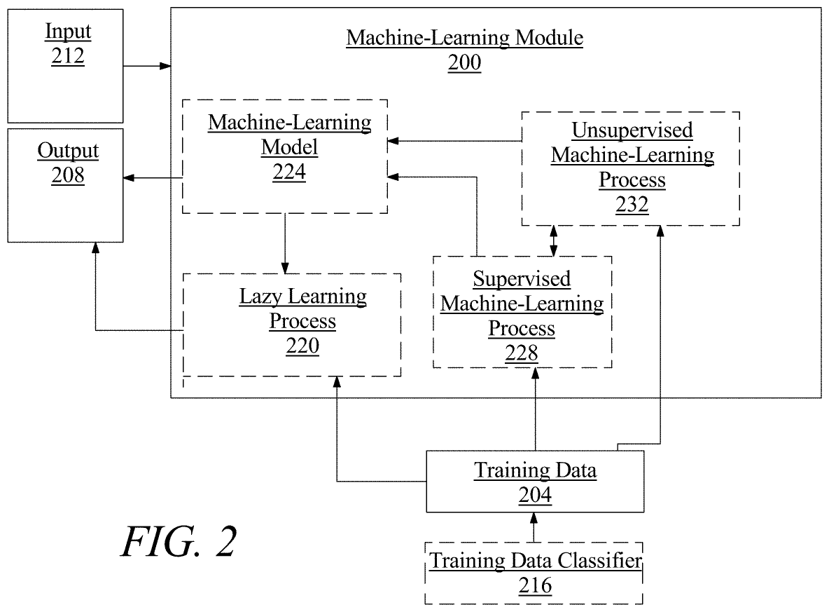

Still referring to FIG. 1, distance may be determined using a distance of and/or used in a classifier as described above in reference to FIG. 2. A classifier used to compute distance may include, without limitation, a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements. KNN algorithm may operate according to any suitable distance, including without limitation vector similarity as described above.

With continued regards to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

Still referring to FIG. 1, according to some embodiments, computing device is configured to receive a ration record 112. As used in this disclosure a “ration record” is a document and/or other collection of data and/or data structure that logs rations historically consumed by a person. For instance, in some embodiments a ration record includes a food log or diary. Alternatively or additionally, in some embodiments, a ration record may include one or more of macro-nutrients or micro-nutrients historically consumed. In some cases, a ration record may contain a precise documentation, alternatively, in some cases the ration record may be noncomprehensive and/or estimated. In some cases, a ration record may be estimated as a function of person's inclusion in a particular culture or tradition. For instance, a person living in Italy may be estimated to have a ration record consistent with a Mediterranean diet, while a person living in Illinois, may be estimated to have a ration record consistent with a Western diet. In some cases, a ration record may include a representation of rations consumed on an ordinary day, for instance a mean number of calories consumed, a mean proportion of calories from fat, a mean proportion of calories from protein, a mean number of calories from carbohydrates, and the like. In some cases, micro-nutrients, such as without limitation vitamins and minerals are documented in a ration record. As used in this disclosure a “ration” is any comestible material, for instance an aliment, a provision, a victual, a sustenance, and the like. Typically, a ration may have nutritional value, for instance it may have caloric value or some other functional value to aid in digestion or sustaining life.

Still referring to FIG. 1, in some embodiments, computing device 104 is further configured to generate, using a ration record 112, at least a change of nutrition 120. A change of nutrition may refer to a change in consumption of one or more of at least a ration or nutrients. For example, in some cases, a change in nutrition may include consuming more or less of a given micro-nutrient or macro-nutrient. Alternatively or additionally, in some cases a change of nutrition 120 may include a change in consumption of at least a ration, for instance increase/decrease consumption of carrots or cheeseburgers. In some cases, one or more machine-learning processes are employed in generating a change of nutrition 120. For instance, generating a change in nutrition 120 may include receiving training data 124, training a machine learning model 128 as a function of a machine learning algorithm 132 and the training data 124, and generating at least a change of nutrition 120 as a function of the machine learning model 128, and the ration record 110. A machine learning model 128 may refer to any machine learning model described in this disclosure; and a machine learning algorithm may refer to any machine learning algorithm 124 used in this disclosure. Further explanation of machine learning processes can be found below, in detail. Training data 124 is also described in greater detail below; and training data 124 may refer to any training data used in this disclosure.

In some cases, and with further reference to FIG. 1, training date 124 correlates nutritional elements to endocrinal representations. As used in this disclosure a “nutritional element” is a representation of nutrients and/or component of diet; for instance, a nutritional element may include a caloric values, micro-nutrient content, macro-nutrient content, certain rations or ration types, and the like. Nutritional elements as included in training data 124 may refer to any of rations, foods, aliments, and the like or nutritional categories, for instance macro-nutrients, micro-nutrients, and the like. Endocrinal representations as included in training data 124 may include any endocrinal representation of an endocrinal state, level, balance, change, function, or the like. In some cases, training data 124 associates known relationships between rations and endocrinal system function. Known relationships between rations and endocrinal representations may be determined, for example from previous intervention of ration consumption, previous nutrition changes, scientific or refereed journal articles, and the like. In some cases, training data 124 may correlate a change of nutrition to a nutritional standard and a ration record. In some embodiment, computing device 104 is additionally configured to generate at least a change of nutrition 120 by generating the at least a change of nutrition 120 as a function of machine learning model 128, desired endocrinal change, and ration record 110. For example, without limitation, machine learning model may be configured to accept as input a desired endocrinal change and a ration record 110 and output a change of nutrition 120. Alternatively or additionally, in some embodiments, computing device 104 is further configured to calculate at least a change of nutrition as a function of a nutritional standard and a ration record. For example, a nutritional standard may be generated as a function of machine learning model 128 and an endocrinal standard; the nutritional standard may, therefore, represent a diet which if adhered to will result in a normal range of endocrinal measures. A change of nutrition 120, may in some embodiments, then be calculated as a function of nutritional standard and ration record 110. For example, a distance between ration record 110 and nutritional standard may be calculated to determine a change in nutrition. Distance between ration record 110 and nutritional standard may be performed according to any method for calculating distance or similarity described in this disclosure.

Still referring to FIG. 1, in some embodiments computing device 104 is configured to output a ration protocol 136 as a function of at least a change of nutrition 120. As used in this disclosure a “ration protocol” is a plan for consuming at least a ration over time, for instance over a predetermined time period, until a desired endpoint has been reached, or indefinitely. In some cases, a ration protocol may include eating at least a ration at certain rate or within a range of rates, such as without limitation once a day, once a week, and the like. In some cases, computing device 104 may be further configured to output a ration protocol 136 by receiving ration classification training data 140, training a ration classification model 144 as a function of a ration classification algorithm 148 and the ration classification training data 140, correlating at least a ration from the ration record to at least a bin of a plurality of bins, as a function of the ration classification model 144 and the ration record 112, selecting a new ration classified to the at least a bin, as a function of the at least a change of nutrition 120, and generating the ration protocol 136, where the ration protocol 136 includes the new ration. In some cases, a new ration may be embody a change of ration when compared to at least a ration from ration record. In some embodiments, comparison of a new ration and at least a ration from ration record may be performed by any calculation described within this disclosure, for example without limitation a distance calculation. In some cases, ration classification training data 140 may correlate a plurality of rations to a plurality of bins. Ration classification training data 140 may include any training data described throughout this disclosure. Likewise, ration classifying model 144 and ration classification algorithm 148 may include any classification models, algorithms, or processes described throughout this disclosure, including but limited to machine-learning process, classifiers, and the like.

Still referring to FIG. 1, in some embodiments, a computing device 104 may be additionally configured to generate at least a change of nutrition 120 by generating a nutrition standard as a function of machine learning model 128 and an endocrinal standard. In some cases, a nutrition standard may include at least a nutritional element which is anticipated to result in a desired endocrine state (i.e., endocrinal standard). A computing device 104 may be configured to classify at least a ration from a ration record 112 to at least a nutritional element, as a function of nutrient classification model and the ration record. Computing device 104 may then be configured to calculate a distance between at least a nutritional element classified to at least a ration and nutrition standard. Distance may be calculated according to any method described throughout this disclosure. Finally, computing device 104 may be configured to generate at least a change of nutrition 120 as a function of distance between nutritional element and nutritional standard. In some cases, nutrient classification model may include a machine-learning model, which may be trained using a training set, such as without limitation nutrient classification training data. In some cases, classification training data may correlate a plurality of rations to a plurality of nutritional elements, for instance without limitation correlating a food item to nutritional information about the food item. Nutrient classification model may be generated by computing device 104 as a function of a nutrient classification algorithm and nutrient classification training data. In some cases, nutrient classification algorithm may include any machine-learning process or algorithm described throughout in this disclosure.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example nutritional elements may be correlated to endocrinal representations, and/or a change of nutrition may be correlated to a nutritional standard and a ration record, and/or a ration may be correlated to one or more bins.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to a plurality of bins. A classifier may include any classifier described throughout this disclosure. In some cases, classification training data may be used to classify at least a ration to at least a bin (or category), such that rations may be grouped together by bin. In some cases, bins may be related to a category of ration for example, vegetable, fruit, starch, meat, fish, and the like. Alternatively or additionally, bins may be demarcated according to a meal or course at which rations grouped within them are consumed, for example breakfast, brunch, lunch, dinner, dessert, and the like. Alternatively or additionally, bins may be demarcated by production type, producer, or ration originator, for example store or ration source. Alternatively or additionally, bins may be demarcated by nutritional elements, for example in some cases rations including similar nutritional elements or like nutrient profiles may be classified together by bin. In some cases, classification may also include generating a probability of classification, for example by way of a Naïve Bayes classification algorithm, as described above.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network including an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include nutritional elements as described above as inputs, endocrinal representations as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

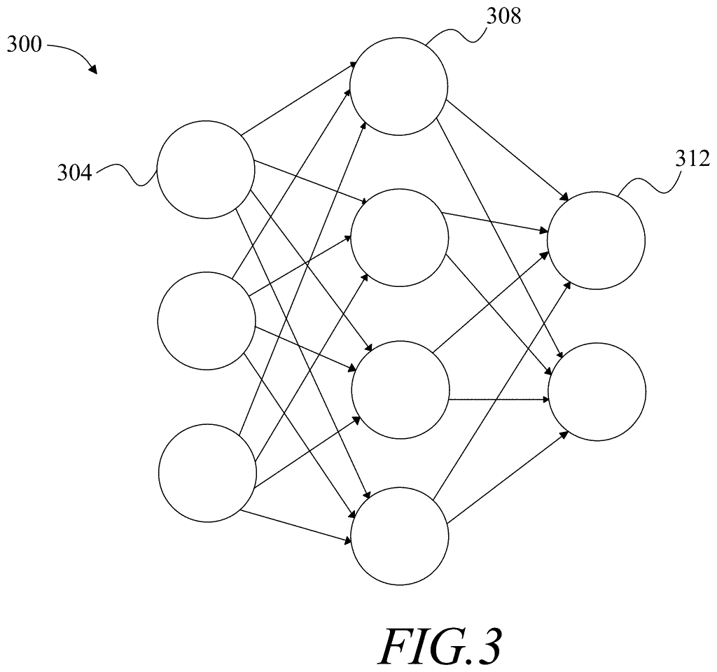

Referring now to FIG. 3 an exemplary embodiment of neural network 300 is illustrated. Neural network also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to input nodes 304, a suitable training algorithm (such as Levenberg-Marquardt, conjugate gradient, simulated annealing, or other algorithms) is then used to adjust the connections and weights between nodes in adjacent layers 308 of the neural network to produce the desired values at output nodes 312. This process is sometimes referred to as deep learning.

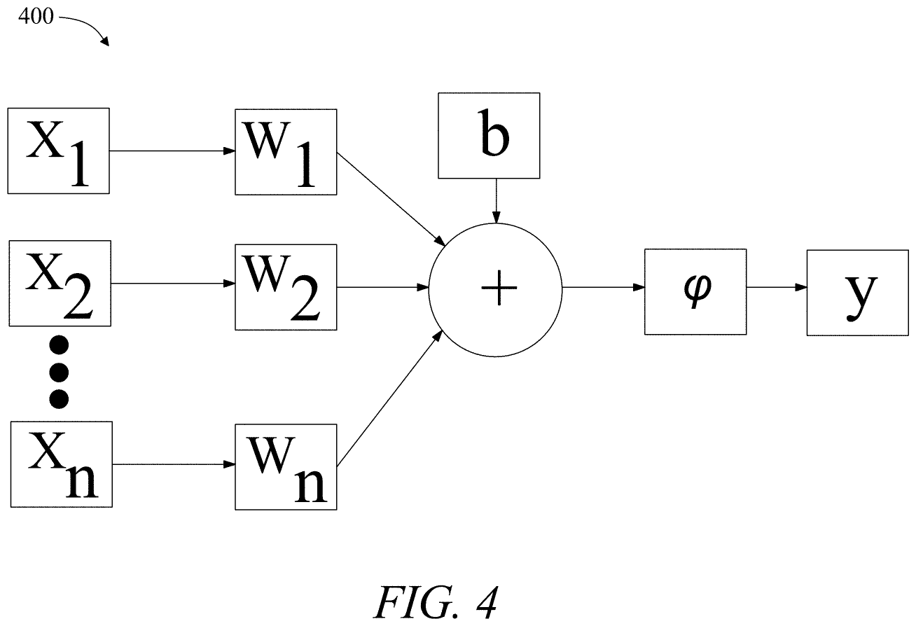

Referring now to FIG. 4, an exemplary embodiment of a node 400 of a neural network is illustrated. A node 400 may include, without limitation a plurality of inputs xᵢ that may receive numerical values from inputs to a neural network containing the node and/or from other nodes. Node 400 may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Still referring to FIG. 4, a neural network may, for example without limitation, receive a desired endocrine change as input and output at least a nutritional element. Additionally or alternatively, a neural network may, for example without limitation, at least a nutritional element as input and output an anticipated endocrinal change. In some cases, a neural network may, for example without limitation, classify at least a nutritional element or a change of nutrition 120 to at least a ration or a ration protocol 136. In some cases, a neural network may additionally output a probability of classification to a predetermined class according to weights wᵢ that are derived using machine-learning processes as described in this disclosure. In some cases, a probability of classification may describe a distance between endocrinal effects anticipated to result from at least a ration or a ration protocol 136 and a desired endocrinal change.

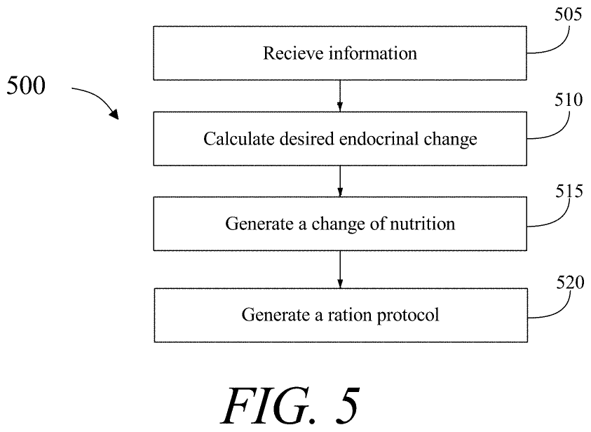

Referring now to FIG. 5, a method 500 of generating a ration protocol for instituting a desired endocrinal change is shown by way of a flowchart. At step 505, a computing device 104 receives information 505; for instance without limitation the information may include at least an endocrinal representation 108 and a ration record 112. An endocrinal representation may include any endocrinal representation described throughout this disclosure, for instance in reference to FIGS. 1-4. A ration record may include any ration record described throughout this disclosure, for instance in reference to FIGS. 1-4.

Continuing in reference to FIG. 5, at step 510, a computing device 104 calculates a desired endocrinal change. In some cases, desired endocrinal change may be calculated as a function of an endocrinal representation 108. Calculating a desired endocrinal change 510 may be performed according to any calculation methods described throughout this disclosure, including without limitation calculating a distance between an endocrinal representation and an endocrinal standard. In some versions, step 510 at calculating desired endocrinal change may additionally include calculating a distance between the endocrinal representation and an endocrinal standard. In some cases, an endocrinal standard may include a normal range of hormone levels. In some cases, step 510 at calculating distance between endocrinal representation and an endocrinal standard additionally includes representing the endocrinal representation as a first vector, representing the endocrinal standard as a second vector, calculating a similarity between the first vector and the second vector, and calculating the distance as a function of the similarity between the first vector and the second vector.

Continuing in reference to FIG. 5, at step 515, a computing device 104 generates at least a change of nutrition 120. In some embodiments, generating a change of nutrition may additionally include receiving training data correlating nutritional elements to endocrinal representations, training a machine learning model as a function of a machine learning algorithm and the training data, and generating the at least a change of nutrition 120 as a function of the machine learning model, and the ration record. Change of nutrition may include any change of nutrition described throughout this disclosure, for example in reference to FIGS. 1-4. Training data may include any training data described throughout this application, for example in reference to FIGS. 1-4. Machine learning model may include any machine learning model described throughout this disclosure, for example in reference to FIGS. 1-4. Machine learning algorithm may include any machine learning algorithm described throughout this disclosure, for example in reference to FIGS. 1-4. In some versions, step 515 at generating at least a change of nutrition additionally includes generating a nutrition standard as a function of a machine learning model and an endocrinal standard, receiving nutrient classification training data correlating a plurality of rations to a plurality of nutritional elements, training a nutrient classification model as a function of a nutrient classification algorithm and the nutrient classification training data, classifying at least a ration from a ration record 112 to at least a nutritional element, as a function of the nutrient classification model and the ration record, calculating a distance between the ration record and the nutrition standard, and generating the at least a change of nutrition 120 as a function of the distance. In some cases, machine learning model may include a convolutional neural network.

Continuing in reference to FIG. 5, at step 520, a computing device 104 outputs a ration protocol 136. According to some embodiments, ration protocol may be output as a function of at least a change of nutrition 120. Ration protocol may include any ration protocol described in this disclosure, for example in reference to FIGS. 1-4. In some versions, outputting a ration protocol additionally includes receiving ration classification training data correlating a plurality of rations to a plurality of bins, training a ration classification model as a function of a ration classification algorithm and the ration classification training data, correlating at least a ration from the ration record to at least a bin of the plurality of bins, as a function of the ration classification model and the ration record, selecting a new ration classified to the at least a bin, as a function of at least a change of nutrition, and outputting the ration protocol, wherein the ration protocol includes the new ration. Ration classification training data may include any training data described throughout this disclosure, for example in reference to FIGS. 1-4. Ration classification model may include any model, or machine learning model, described throughout this disclosure, for example in reference to FIGS. 1-4. Ration classification algorithm may include any algorithm, or machine learning algorithm, described throughout this disclosure, for example in reference to FIGS. 1-4. In some versions, step 820 at classifying the at least a ration to the at least a bin, additionally includes generating a probability of classification. Probability of classification may include any probability of classification described throughout this disclosure, for example in reference to FIGS. 1-4.

Still referring to FIG. 5, in some embodiments, step 515 at generating at least a change of nutrition may additionally include generating the at least a change of nutrition as a function of a machine learning model, a desired endocrinal change, and a ration record.

Still referring to FIG. 5, in some embodiments, step 520 at outputting a ration protocol 136 additionally includes receiving ration classification training data correlating a plurality of rations to a plurality of bins, training a ration classification model as a function of a ration classification algorithm and the ration classification training data, classifying at least a ration from ration record 112 to at least a bin of to the plurality of bins, as a function of the ration classification model and the ration record, selecting a new ration classified to the at least a bin, as a function of at least a change of nutrition 136, and outputting the ration protocol 136. In some cases, ration protocol 136 may include a new ration.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

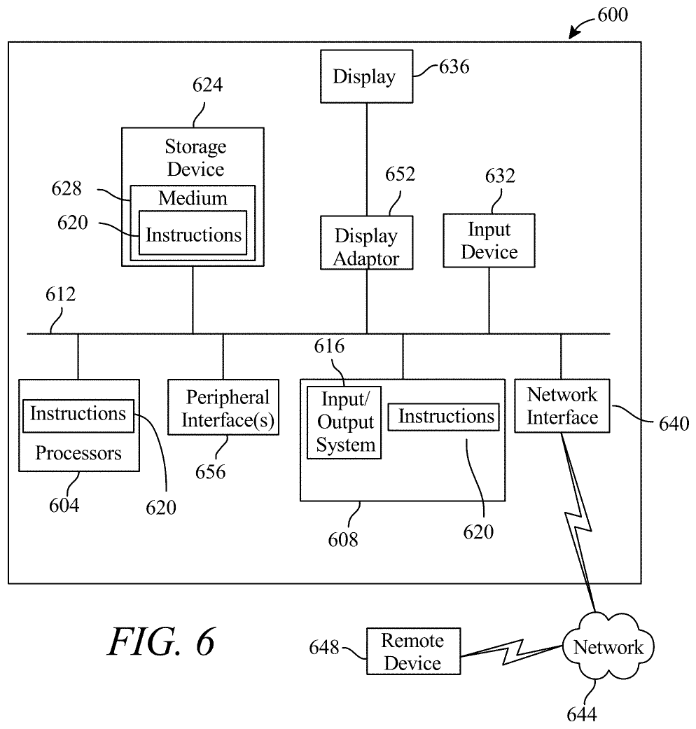

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of generating a ration protocol for instituting a desired endocrinal change comprising:
receiving, using a computing device, at least an endocrinal representation and a ration record;
calculating, using the computing device, a desired endocrinal change as a function of the at least an endocrinal representation;
generating, using the computing device and the ration record, at least a change of nutrition, wherein generating the at least a change of nutrition further comprises:
receiving training data correlating nutritional elements to endocrinal representations;
training a machine learning model as a function of a machine learning algorithm and the training data; and
generating at least a change of nutrition as a function of the machine learning model, and the ration record; and
outputting, using the computing device, the ration protocol as a function of the at least a change of nutrition.

2. The method of claim 1 wherein outputting the ration protocol further comprises:
receiving ration classification training data correlating a plurality of rations to a plurality of bins;
training, using the computing device, a ration classification model as a function of a ration classification algorithm and the ration classification training data;
classifying, using the computing device, at least a ration from the ration record to at least a bin of the plurality of bins, as a function of the ration classification model and the ration record;
selecting, using the computing device, a new ration classified to the at least a bin, as a function of the at least a change of nutrition; and
outputting, using the computing device, the ration protocol, wherein the ration protocol comprises the new ration.

3. The method of claim 2, classifying the at least a ration to the at least a bin further comprises generating a probability of classification.

4. The method of claim 1, wherein calculating the desired endocrinal change further comprises calculating a distance between the endocrinal representation and an endocrinal standard.

5. The method of claim 4, wherein the endocrinal standard comprises a normal range of hormone levels.

6. The method of claim 4, wherein calculating the distance between the endocrinal representation and the endocrinal standard further comprises:
representing the endocrinal representation as a first vector;
representing the endocrinal standard as a second vector;
calculating a similarity between the first vector and the second vector; and
calculating the distance as a function of the similarity between the first vector and the second vector.

7. The method of claim 1, wherein generating the at least a change of nutrition further comprises:
generating the at least a change of nutrition as a function of the machine learning model, the desired endocrinal change, and the at least a ration record.

8. The method of claim 1, wherein generating the at least a change of nutrition further comprises:
generating a nutrition standard as a function of the machine learning model and an endocrinal standard;
calculating a distance between the ration record and the nutrition standard; and
generating the at least a change of nutrition as a function of the distance.

9. The method of claim 8 wherein outputting the ration protocol further comprises:
receiving ration classification training data correlating a plurality of rations to a plurality of bins;
classifying, using the computing device, at least a ration from the ration record to at least a bin of to the plurality of bins, as a function of a ration classification model and the ration record;
selecting, using the computing device, a new ration classified to the at least a bin, as a function of the at least a change of nutrition; and
outputting, using the computing device, the ration protocol, wherein the ration protocol comprises the new ration.

10. The method of claim 1, wherein the machine learning model comprises a convolutional neural network.

11. A system for generating a ration protocol for instituting a desired endocrinal change comprising a computing device configured to:
receive at least an endocrinal representation and a ration record;
calculate the desired endocrinal change as a function of the at least an endocrinal representation;
generate, using the ration record, at least a change of nutrition, wherein generating the at least a change of nutrition, further comprises:
receiving training data correlating nutritional elements to endocrinal representations;
training a machine learning model as a function of a machine learning algorithm and the training data; and
generating at least a change of nutrition as a function of the machine learning model, and the ration record; and
output the ration protocol as a function of the at least a change of nutrition.

12. The system of claim 11 wherein outputting the ration protocol further comprises:
receiving ration classification training data correlating a plurality of rations to a plurality of bins;
training a ration classification model as a function of a ration classification algorithm and the ration classification training data;
classifying at least a ration from the ration record to at least a bin of the plurality of bins, as a function of the ration classification model and the ration record;
selecting a new ration classified to the at least a bin, as a function of the at least a change of nutrition; and
outputting the ration protocol, wherein the ration protocol comprises the new ration.

13. The system of claim 12, wherein classifying the at least a ration to the at least a bin further comprises generating a probability of classification.

14. The system of claim 11, wherein calculating the desired endocrinal change further comprises calculating a distance between the endocrinal representation and an endocrinal standard.

15. The system of claim 14, wherein the endocrinal standard comprises a normal range of hormone levels.

16. The system of claim 14, wherein calculating the distance between the endocrinal representation and the endocrinal standard further comprises:
representing the endocrinal representation as a first vector;
representing the endocrinal standard as a second vector;
calculating a similarity between the first vector and the second vector; and
calculating the distance as a function of the similarity between the first vector and the second vector.

17. The system of claim 11, wherein generating the at least a change of nutrition further comprises:
generating the at least a change of nutrition as a function of the machine learning model, the desired endocrinal change, and the ration record.

18. The system of claim 11, wherein generating the at least a change of nutrition further comprises:
generating a nutrition standard as a function of the machine learning model and an endocrinal standard;
calculating a distance between the ration record and the nutrition standard; and
generating the at least a change of nutrition as a function of the distance.

19. The system of claim 18 wherein outputting the ration protocol further comprises:
receiving ration classification training data correlating a plurality of rations to a plurality of bins;
classifying, using the computing device, at least ration from the ration record to at least a bin of the plurality of bins, as a function of a ration classification model and the ration record;
selecting, using the computing device, a new ration classified to the at least a bin, as a function of the at least a change of nutrition; and
outputting, using the computing device, the ration protocol, wherein the ration protocol comprises the new ration.

20. The system of claim 11 wherein the machine learning model comprises a convolutional neural network.
